(19) United States
(12) Patent Application Publication
(10) Pub. No.: US 2020/0354229 A1
(43) Pub. Date: Nov. 12, 2020

(54) HYDROTHERMAL SYNTHESIS OF THE MOLYBDENUM DIOXIDE NANOPARTICLES DIRECTLY ONTO A METAL SUBSTRATE

(71) Applicant: University of South Florida, Tampa, FL (US)

(72) Inventors: Michael Scott McCrory, Coral Springs, FL (US); Manoj Kumar Ram, Palm Harbor, FL (US)

(21) Appl. No.: 16/936,596

(22) Filed: Jul. 23, 2020

Related U.S. Application Data
(63) Continuation of application No. PCT/US2019/022321, filed on Mar. 14, 2019.
(60) Provisional application No. 62/642,831, filed on Mar. 14, 2018.

(51) Int. Cl. C01G 39/02; B01J 23/28; B01J 23/72; B01J 35/00; B01J 37/04; B01J 37/16; B01J 37/02; A61L 2/10; A61L 2/08; A61L 9/20; H01M 4/48; H01M 4/66; H01M 10/0525; C02F 1/32; C02F 1/72; B01D 53/00; B01D 53/86; H01G 11/46

(52) U.S. Cl. CPC C01G 39/02; H01M 2004/027; B01J 23/72; B01J 35/0013; B01J 35/004; B01J 37/04; B01J 37/16; B01J 37/0225; A61L 2/10; A61L 2/088; A61L 9/205; H01M 4/483; H01M 4/661; H01M 10/0525; C02F 1/32; C02F 1/725; B01D 53/007; B01D 53/8668; B01D 53/8609; B01D 53/8628; B01D 53/864; H01G 11/46; C01P 2004/64; C01P 2006/40; C01P 2004/03; C01P 2002/72; B01J 23/28

(57) ABSTRACT

Provided are a method of synthesizing molybdenum dioxide (MoO₂) directly onto a metal substrate to form a coating on the surface of the substrate, products having a coated metal surface produced by the disclosed method, and their uses to decontaminate water and/or air. The coated metal surface disclosed herein also may be used as a structural component of a Li-ion battery, a supercapacitor, or a sensor for detecting a molecule.

### CROSS-REFERENCE TO RELATED APPLICATIONS

This application is a continuation of and claims priority to International Patent Application No. PCT/US2019/022321, entitled “Hydrothermal Synthesis of Molybdenum Dioxide Nanoparticles Directly onto a Metal Substrate”, filed Mar. 14, 2019 which claims the benefit of U.S. Provisional Patent Application No. 62/642,831, entitled “Hydrothermal Synthesis of MoO₂ Nanoparticles Directly onto a Copper Substrate and Their Ability to Decontaminate Water”, filed Mar. 14, 2018, the contents of each of which are hereby incorporated by reference into this disclosure.

### TECHNICAL FIELD

This disclosure relates to methods for synthesizing molybdenum dioxide (MoO₂) directly onto a substrate, such as a copper substrate. The product of the present methods may be used to decontaminate water and/or air.

### BACKGROUND OF THE INVENTION

Most decontamination of water by a photocatalyst is done in what is called slurry. In these cases, the active material used to decontaminate the solution is mixed directly into the solution to allow the reaction to occur. Since the mixture is continually mixing, the active material is almost always in contact with the pollutant, allowing for maximum effectiveness. The downside to this process is that once the active material has decontaminated the original pollutant in the water, the active material is now a pollutant of its own that must be removed from the water by filtration, centrifugation, or other means. There remain a need for photocatalysts that may be used to decontaminate water with high effectiveness, while generating minimum pollution.

Current techniques for preparing MoO₂ coating on metal surfaces may take multiple steps. For example, anode materials for Li-ion batteries currently are synthesized alone, and must then be coated onto a current collector in a spate step. The coating usually involves mixing the active material into a slurry, with a solvent and a binder material, then coating the slurry onto a current collector (usually copper for anode materials) and then heated in an oven to drive out the solvent. There remains a need for more succinct approach for the coating process.

### SUMMARY OF THE INVENTION

In one aspect, provided is a method of synthesizing MoO₂ nanoparticles, comprising mixing MoO₃, a metal substrate, and a reducing agent in water to form a mixture; and heating the mixture, whereby MoO₂ nanoparticles are produced as a coating on the surface of the metal substrate.

In another aspect, provided is a product, which includes a metal surface coated by MoO₂ nanoparticles by a method as described herein. The product may be useful in decontaminating water or air, for example, by facilitating the conversion of pollutant materials into carbon dioxide, water or small molecules. In some embodiments, the MoO₂ coated metal surface as described herein may be useful as a structural component of a Li-ion battery, a supercapacitor, or a sensor for detecting a molecule.

Other aspects of the invention will become apparent by consideration of the detailed description and accompanying drawings.

### BRIEF DESCRIPTION OF THE DRAWINGS

FIG. 1 is a step-by-step schematic of the typical methylene blue (MB) degradation experiment, sample collection and analysis process for the MoO₂ coated copper samples.

FIG. 2 schematically illustrates a formation mechanism of MoO₂ nanoparticles onto a copper substrate.

FIG. 3 is SEM image of the MoO₃ precursor.

FIG. 4 illustrates SEM images of the MoO₂ coated copper.

FIG. 5 illustrates XRD patterns for a) copper substrate, b) MoO₃, c) MoO₂, and d) MoO₂ coated copper.

FIG. 6 graphically illustrates concentration (C/Co) vs. time (min.) for the decontamination of 10 mL MB by the MoO₂ coated copper substrate.

### DETAILED DESCRIPTION

The present disclosure relates to a method of synthesizing MoO₂ nanoparticles directly onto a metal substrate, such that the MoO₂ nanoparticles form a coating on the surface of the metal substrate. As an example, a process is described herein to synthesize MoO₂ from MoO₃ directly onto a copper substrate. In particular embodiments, the process herein does not use any binder material. The process may involve a single step, hydrothermal synthesis technique to coat a metal substrate (such as copper) with MoO₂ nanoparticles.

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” as used herein as applied to one or more values of interest, refers to a value that is similar to a stated reference value. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

### 2. Method of Synthesizing MoO₂ Nanoparticles

In one aspect, the present disclosure relates to a method of synthesizing MoO₂ nanoparticles, comprising

mixing MoO₃, a metal substrate, and a reducing agent in water to form a mixture; and

heating the mixture, whereby MoO₂ nanoparticles are produced as a coating on the surface of the metal substrate.

Suitable MoO₃ materials include commercially available products and MoO₃ compound produced by any method known in the art. In some embodiments, the MoO₃ is produced by heating ammonium molybdate at about 350° C.

Suitable reducing agent includes any organic or inorganic reagent capable of reacting with MoO₃ to form MoO₂. In some embodiments, the reducing agent comprises an organic molecule, such as an oxygen-containing ligand. In some embodiments, the reducing agent is ethylene glycol.

The metal substrate may include a metal or an alloy of metals. In some embodiments, the metal substrate may include copper, aluminum, nickel, titanium, steel, or a combination thereof. In some embodiments, the metal substrate includes copper or copper alloy. In particular embodiments, the metal substrate includes copper. The metal may be present in the metal substrate at an amount of at least 90%, at least 95%, or at least 99% by weight. The metal substrate may be in a form of a sheet or chip having a thickness of about 1 mm or less, such as a thickness of about 0.5 mm, about 0.25 mm, or about 0.1 mm. For example, the metal substrate may be a copper sheet (99% by weight) having a thickness of about 0.25 mm.

The mixture may be heated under any condition necessary based on the starting source for the MoO₃ and/or the reducing agent being used. In some embodiments, the mixture is heated under a pressure greater than about 1 atm. In some embodiments, the mixture is heated under a pressure greater than about 10 atm, greater than about 20 atm, greater than about 30 atm, greater than about 40 atm, greater than about 50 atm, greater than about 60 atm, greater than about 70 atm, greater than about 80 atm, greater than about 90 atm, or greater than about 100 atm. In some embodiments, the mixture is heated under a pressure of about 2 atm to about 100 atm, such as about 2 atm to about 90 atm, about 2 atm to about 80 atm, about 2 atm to about 70 atm, about 2 atm to about 60 atm, about 2 atm to about 50 atm, about 2 atm to about 40 atm, about 2 atm to about 30 atm, about 2 atm to about 20 atm, about 2 atm to about 10 atm. In particular embodiments, the pressure is at about 2 atm to about 50 atm.

In some embodiments, the mixture is heated to a temperature between about 100° C. and about 200° C., between about 100° C. and about 250° C., between about 100° C. and about 300° C., between about 100° C. and about 400° C., between about 100° C. and about 500° C., between about 150° C. and about 200° C., between about 150° C. and about 250° C., between about 150° C. and about 300° C., between about 150° C. and about 400° C., or between about 150° C. and about 500° C. In some embodiments, the mixture is heated to about 150° C., about 160° C., about 170° C., about 180° C., about 190° C., about 200° C., about 210° C., or about 220° C. In particular embodiments, the mixture is heated to about 180° C. The temperature may be maintained at a specific value or range during the heating process.

In some embodiments, the mixture is heated for less than or equal to about 20 hours. In some embodiments, the mixture is heated for less than or equal to about 20 hours, less than or equal to about 15 hours, less than or equal to about 12 hours, less than or equal to about 10 hours, less than or equal to about 8 hours, less than or equal to about 6 hours, less than or equal to about 4 hours, or less than or equal to about 2 hours. In some embodiments, the mixture is heated for about 15 hours or less, about 12 hours or less, or about 10 hours or less. In some embodiments, the mixture is heated for about 8 hours, about 9 hours, about 10 hours, about 11 hours, about 12 hours, about 13 hours, about 14 hours, or about 15 hours. In particular embodiments, the mixture is heated for about 12 hours.

In some embodiments, the mixture is heated under a pressure of about 2 atm to about 50 atm (including, for example, about 2 atm to about 30 atm) to a temperature of about 100° C. to about 250° C. (including, for example, about 150° C. and about 200° C., more particularly about 180° C.), and the heating is maintained for a period of about 12 hours or less (including, for example, about 12 hours or about 10 hours).

In some embodiments, the present method does not involve the use of any binder materials known in the art. Such binder materials may include, for example, those providing adhesion between metal oxide and metal surfaces. In some embodiments, the present method does not involve the use of metal or metalloid oxide (e.g., alumina, silica or yttria), inorganic salts (e.g., magnesium and potassium silicates), or organic polymers (e.g., acrylics, epoxides, styrenes, polyurethanes, haloalkylenes, and various copolymers, such as styrene-acrylates and styrene-butadienes) as binders.

The present method may include additional steps to provide a ready-to-use product. The additional steps may include, for example, allowing the heated mixture to cool to room temperature, retrieving the coated metal substrate, cleaning the coated metal substrate, and drying the coated metal substrate.

In some embodiments, the method includes retrieving the coated metal substrate from the mixture after the heating step. The retrieval may be done using any known method in the art, for example, manual separation, filtration, sedimentation, or centrifugation.

In some embodiments, the method includes cleaning the coated metal substrate. The cleaning may include rinsing the substrate with a solvent. The solvent may include, for example, deionized water, alcohol (such as ethanol), and mixtures thereof. In some embodiments, the method includes cleaning the coated substrate with ethanol and after the heating step. In some embodiments, the method further comprises cleaning the coated substrate with water after the heating step. In some embodiments, the method further comprises cleaning the coated substrate with ethanol and water after the heating step.

In some embodiments, the method includes drying the coated substrate after the heating step. The drying may be accomplished by any known method in the art, including, for example, air drying, vacuum drying, and oven drying. In exemplary embodiments, the method further comprises drying the mixture in an oven after the heating step.

The present disclosure provides an advantageous coating method for MoO₂ over the conventional technology, in which the synthesis of coating material and the coating process are separate processes. For example, for Li-ion batteries, currently anode materials (MnO₂, MoO₂, TiO₂, NiO₂, MoO₂, etc.) are synthesized alone, and must then be coated onto a current collector in a spate step. The coating usually involves mixing the active material into a slurry, with a solvent, and a binder material, then coating the slurry onto a current collector (usually copper for anode materials) and then heated in an oven to drive out the solvent. In contrast, the present method may prepare the MoO₂ coating on a metal substrate in a one-step, hydrothermal process, in which MoO₂ nanoparticles may be synthesized directly onto the metal surface.

### 3. MoO₂ Nanoparticles and MoO₂ Coated Metal Surface

The MoO₂ nanoparticles produced by the present method may have a diameter of about 50 nm, about 40 nm, about 30 nm, about 20 nm, or about 10 nm. The diameter of the MoO₂ nanoparticles may be in a range of about 10 nm to about 50 nm, about 20 nm to about 50 nm, about 30 nm to about 50 nm, or about 40 nm to about 50 nm. In particular embodiments, the diameter of the MoO₂ nanoparticles is about 30 nm to about 50 nm.

In some embodiments, the MoO₂ coated metal surface does not comprise MoO₃.

In some embodiment, the properties of the MoO₂ nanoparticles and MoO₂ coated metal surface disclosed herein may be characterized by known techniques, such as X-ray diffraction (XRD) and electron microscope (SEM). In some embodiments, XRD may be used to confirm that all of the MoO₃ had been reduced to MoO₂, and/or that no other compounds had formed between the molybdenum and surface of the metal substrate (e.g., copper). In some embodiments, SEM images of the MoO₂ coated metal substrate may be obtained, from which the diameter of the MoO₂ nanoparticles may be determined.

In some embodiments, the MoO₂ nanoparticle may exhibit adsorbent properties. In some embodiments, the MoO₂ nanoparticle may exhibit photocatalytic properties. In some embodiments, the MoO₂ nanoparticle may simultaneously exhibit adsorbent and photocatalytic properties.

In some embodiments, the present disclosure provides a product, which includes a metal surface coated by MoO₂ nanoparticles according to the method disclosed herein. The metal surface may be coated by the method disclosed herein before or after the metal surface is incorporated in the product. The products may include, but are not limited to, decontaminants, batteries, sensors, or electronic devices.

In some embodiments, the MoO₂ coated metal surface herein forms at least a part of an anode of a Li-ion battery. For example, the present method may be used to provide a coating of MoO₂ nanoparticles on the anode of a Li-ion battery. Advantageously, the metal surface of the anode may be coated with MoO₂ nanoparticles without any binder in a one-step hydrothermal process as disclosed herein.

In some embodiments, the MoO₂ coated metal surface herein forms at least a part of a symmetric or asymmetric electrode of a supercapacitor. For example, the present method may be used to provide a coating of MoO₂ nanoparticles on a metal surface of a symmetric or asymmetric electrode of a supercapacitor. Advantageously, the metal surface of the supercapacitor may be coated with MoO₂ nanoparticles without any binder in a one-step hydrothermal process as disclosed herein.

In some embodiments, the MoO₂ coated metal surface herein forms at least a part of a sensor for detecting a molecule selected from the group consisting of nitrogen oxide (e.g., NO and NO₂), nitrous oxide (N₂O), alcohol (e.g., ethanol), sulphur oxide (e.g., SO, SO₂, SO₃, S₇O₂, S₆O₂, and S₂O₂), and a combination thereof. For example, the present method may be used to provide a coating of MoO₂ nanoparticles on a metal surface of a sensor, such as exhaust gas sensors known in the art for detecting the above molecules. Advantageously, the metal surface of the sensor may be coated with MoO₂ nanoparticles without any binder in a one-step hydrothermal process as disclosed herein.

### 4. Method of Decontamination

The present disclosure also provides a method of decontaminating water or air using a product as disclosed herein. The water or air may be contaminated by a pollutant. The present products may reduce the amount of the pollutant (by weight or by volume) in the contaminated water or air. For example, the present products may chemically react with the pollutant to at least partially remove the pollutant from the water or air.

In some embodiments, provided is a method of decontaminating water, comprising contacting contaminated water comprising a water pollutant with a product having a metal surface coated with MoO₂ nanoparticles, as disclosed herein, thereby reducing the amount of the water pollutant.

The contaminated water may include any fluid containing water, such as an aqueous solution or suspension. Examples of contaminated water include, but are not limited to, industrial or household waste water, lake water, or sea water.

The water pollutant may include an organic compound, a biological contaminant, or a combination thereof.

Examples of the organic compounds may include, but are not limited to aliphatic organic compounds (such as alkanes, alkenes, alkynes), halogenated compounds (such as chloroform), ketones, aldehydes, organic alcohols, organic acid and substituted organic acid (such as carboxylic acids and chloro-trichloroacetic acid), ethers, phenol based compounds (such as 4-chlorophenol and pentachlorophenol), benzene and derivatives (such as chlorobenzene and 4-chlorotoluene), polyaromatic compounds, and combinations thereof.

In some embodiments, the water pollutant comprises an organic compound selected from the group consisting of chloroform, alcohols, trichloroacetic acid, 4-chlorophenol, pentachlorophenol, benzene, chlorobenzene, 4-chlorotoluene, or a combination thereof.

In some embodiments, the organic compounds may be a dye or a chemical toxin. The dye may be an acid dye, including, for example, anthraquinone type, azo dye type, and triphenylmethane type. The dye may be a basic dye, including, for example, methylene blue dyes and crystal violet dyes, etc. The dye may be a substantive dye, including, for example, trypan blue, direct blue. The dye may be a disperse dye, including, for example, disperse yellow 26, disperse red 1, or disperse orange 37, anthraquinone molecule with nitro, amine, or hydroxyl. The dye may be a sulfur dye, including, for example, sulfur black 1. The dye may be a vat dye, including, for example, vat red 10, vat violet 13, and vat orange 1. The dye may be a reactive dye, including, for example, 1, 1 bi- and polyfunctional reactive dyes. The dye may be an azo dye, including, for example, methyl red, methyl orange, and congo red. The dye may be an aniline dye, including, for example, Perkin's mauve (aniline violet), fuchsin, methyl green, aniline blue, and magenta (aniline red). The dye may be a pigment dyes, a mordant dye (such as mordant red 19), a naphthol dye, a phthalocyanine dye, a xanthene dyes, or a pyronin dye. The dye may be an anthraquinone dye or a dye derived of anthraquinone. The dye may be a rhodamine dye or a derivative of rhodamine. The dye may be a fluorine dye or a fluorine based dye. In some embodiments, the water pollutant includes one or more dyes. In particular embodiments, the water pollutant includes methylene blue (MB).

The chemical toxin may be a chemical warfare agent. Examples of chemical warfare agents may include, but are not limited to, nerve agents (such as sarin, cyclohexylsarin, soman, tabun, and VX), choking agents (such as chlorine, phosgene, and diphosgene), and blistering agents (such as vesicants, sulfur mustards, arsenicals or urticants).

The biological contaminant may be a protein, a bacterium, or a combination thereof. Examples of proteins include various polypeptides and enzymes. Examples of bacteria include, but are not limited, various foodborne bacteria such as E. coli, Campylobacter jejuni (which can lead to secondary Guillain-Barré syndrome and periodontitis), Clostridium perfringens (the “cafeteria germ”), and Salmonella spp. (its S. typhimurium infection is caused by consumption of eggs or poultry that are not adequately cooked or by other interactive human-animal pathogens) The bacteria may be Escherichia coli O157:H7 enterohemorrhagic (EHEC) which can cause hemolytic-uremic syndrome. The bacteria may be Bacillus cereus, Escherichia coli (other virulence properties, such as enteroinvasive (EIEC), enteropathogenic (EPEC), enterotoxigenic (ETEC), enteroaggregative (EAEC or EAgEC)), Listeria monocytogenes, Shigella spp., Staphylococcus aureus, Staphylococcal enteritis, Streptococcus, Vibrio cholera (including 01 and non-01), Vibrio parahaemolyticus, Vibrio vulnificus, Yersinia enterocolitica, and Yersinia pseudotuberculosis.

In some embodiments, the water pollutant comprises a bacterium, which is selected from the group consisting of E. coli, Campylobacter jejuni, Clostridium perfringens, Salmonella spp., Bacillus cereus, Listeria monocytogenes, Shigella spp., Staphylococcus aureus, Staphylococcal enteritis, Streptococcus, Vibrio cholera, Vibrio parahaemolyticus, Vibrio vulnificus, Yersinia enterocolitica, Yersinia pseudotuberculosis, and a combination thereof. In some embodiments, the water pollutant comprises a bacterium, which is E. coli, Salmonella spp., or a combination thereof.

In some embodiments, provided is a method of decontaminating air, comprising contacting contaminated air comprising an air pollutant with a product having a metal surface coated with MoO₂ nanoparticles, as disclosed herein, thereby reducing the amount of the air pollutant.

The air pollutant may include, for example, an organic gas, an inorganic gas, a biotoxin, or a combination thereof.

Examples of organic gas include, but are not limited to, volatile organic compounds such as volatile alcohol, ketone, acetone, ethers, phenol, etc.

Examples of inorganic gas include, but are not limited to, sulphur oxide (such as SO₂), nitrogen oxide (such as NO₂), carbon monoxide.

Examples of biotoxins include, but are not limited to Abrin, Brevetoxin, Colchicine, Digitalis, Nicotine, Ricin, Saxitoxin, Strychnine, Tetrodotoxin, and Trichothecene.

Air pollutant may also include those causing airborne diseases or sickness, such as mold (e.g., Cladosporium, Penicillium, Alternaria, Aspergillus), bacteria (e.g., E. coli and Salmonella spp.); and virus (e.g., flu virus, rhinovirus, mumps, and measles virus).

The methods disclosed herein may further include comprising carrying out a photocatalytic reaction of the water pollutant or air pollutant catalyzed by the MoO₂ coated surface under UV to visible light. The method may further include absorbing the water pollutant or air pollutant onto the MoO₂ coated surface and oxidizing the water pollutant into carbon dioxide, water or a small molecule

In some embodiments, the method further comprises applying visible or UV light. In some embodiments, the visible or UV light may be applied with an intensity equal to or greater than about 800 W/m². In some embodiments, the visible light may have an intensity between about 700 W/m² and 900 W/m², between about 600 W/m² and 1000 W/m², between about 500 W/m² and 1100 W/m², between about 400 W/m² and 1200 W/m², between about 700 W/m² and 1000 W/m², between about 700 W/m² and 1100 W/m², between about 700 W/m² and 1200 W/m², between about 600 W/m² and 900 W/m², between about 500 W/m² and 900 W/m², or between about 400 W/m² and 900 W/m².

In some embodiments, a product containing about 0.5 mg to about 10 mg of MoO₂ nanoparticles as disclosed herein may be used to remove at least 50.0% of a pollutant in contaminated water or air in less than 10 minutes in the presence of visible light. In some embodiments, a product containing about 0.5 mg to about 10 mg, about 0.5 mg to about 5.0 mg, about 0.5 mg to about 4.0 mg, or about 0.5 mg to about 3.0 mg, or about 0.5 mg to about 2.0 mg of MoO₂ nanoparticles as disclosed herein is used to remove at least 50.0%, at least 60.0%, at least 70.0%, at least 75.0%, at least 80.0%, at least 85.0%, at least 90.0%, or at least 95.0% of a pollutant in contaminated water or air in less than 10 minutes in the presence of visible light. In some embodiments, the product may be used to remove at least 75.0%, at least 80.0%, at least 85.0%, at least 90.0%, at least 95.0%, at least 96.0%, at least 97.0%, at least 98.0%, at least 99.0%, at least 99.5%, or at least 99.9% of a pollutant in less than about 10 minutes, less than about 5 minutes, less than about 4 minutes, less than about 3 minutes, less than about 2 minutes, less than about 1 minute, or less than about 30 seconds in the presence of visible light.

In some embodiments, a product containing about 0.5 mg and 10 mg of MoO₂ nanoparticles as disclosed herein may be used to remove at least 70.0% of methylene blue (MB) from 10 mL of a 10 mg/L MB aqueous solution in 10 minutes or less in the presence of visible light. In some embodiments, a product containing about 0.5 mg and 5.0 mg of MoO₂ nanoparticles as disclosed herein may be used to remove at least 50% of methylene blue (MB) from 10 mL of a 10 mg/L MB aqueous solution in 5 minutes or less in the presence of visible light.

In some embodiments, the method does not comprise providing or applying visible or UV light. In some embodiments, a product containing about 0.5 mg and 10 mg of MoO₂ nanoparticles as disclosed herein may be used to remove at least 50.0% of a pollutant in contaminated water or air in less than 10 minutes in the absence of visible light and UV light. In some embodiments, a product containing about 0.5 mg to about 10 mg, about 0.5 mg to about 5.0 mg, about 0.5 mg to about 4.0 mg, about 0.5 mg to about 3.0 mg, or about 0.5 mg to about 2.0 mg of MoO₂ nanoparticles as disclosed herein is used to remove at least 50.0%, at least 60.0%, at least 70.0%, at least 75.0%, at least 80.0%, at least 85.0%, at least 90.0%, or at least 95.0% of a pollutant in contaminated water or air in less than 10 minutes in the absence of visible light and UV light. In some embodiments, the product may be used to remove at least 75.0%, at least 80.0%, at least 85.0%, at least 90.0%, at least 95.0%, at least 96.0%, at least 97.0%, at least 98.0%, at least 99.0%, at least 99.5%, or at least 99.9% of a pollutant in less than about 10 minutes, less than about 5 minutes, less than about 4 minutes, less than about 3 minutes, less than about 2 minutes, less than about 1 minute, or less than about 30 seconds in the absence of visible light and UV light.

In some embodiments, a product containing about 0.5 mg and 10 mg of MoO₂ nanoparticles as disclosed herein may be used to remove at least 50.0% of methylene blue (MB) from 10 mL of a 10 mg/L MB aqueous solution in 10 minutes or less in the absence of visible light. In some embodiments, a product containing about 0.5 mg and 5.0 mg of MoO₂ nanoparticles as disclosed herein may be used to remove at least 50% of methylene blue (MB) from 10 mL of a 10 mg/L MB aqueous solution in 5 minute or less in the absence of visible light.

In some embodiments, the pollutant (such as a dye) may be broken down into CO₂, H2O, and/or other chemical byproducts. The pollutant may be broken down by a variety of mechanisms known in the art. In some embodiments, the MoO₂ nanoparticle forms an electron-hole pair with the pollutant, and/or the product coated with MoO₂ nanoparticles chemically reacts with the pollutant to produce radical intermediates. In some embodiments, MoO₂ nanoparticles coated on porous substrates may allow the contaminant to have maximum contact with the nanomaterial to decontaminate 100%.

In a particular embodiment, a process is described herein to synthesize MoO₂ directly onto a copper substrate, with no binder material, in a single step hydrothermal reaction. It is believed to be the first report of such a synthesis method. All of the MoO₃ may be reduced to MoO₂, and no other compounds are formed between the molybdenum and copper, as confirmed by XRD. The MoO₂ coated copper substrate may have uniform nanoparticles ranging from about 30 to about 50 nm, as shown by the SEM images. The MoO₂ coated copper substrate may decontaminate over 50% of the methylene blue (MB) from water in 10 minutes without exposure to light, while it may decontaminate over 70% of the MB from water in 10 minutes with exposure to light.

### EXAMPLES

Materials and Methods. All materials were purchased from Sigma Aldrich and used without any modification unless otherwise noted. X-ray diffraction (XRD) measurements were performed using a PANalytical X'Pert PRO diffractometer with Cu Kα radiation (λ=1.5406 Å). Scanning electron microscope (SEM) measurements were performed using a Hitachi SU-70 ultra-high resolution scanning electron microscope. UV-visible spectrophotometry was measured using a Jasco J-530 UV-Vis Spectrophotometer.

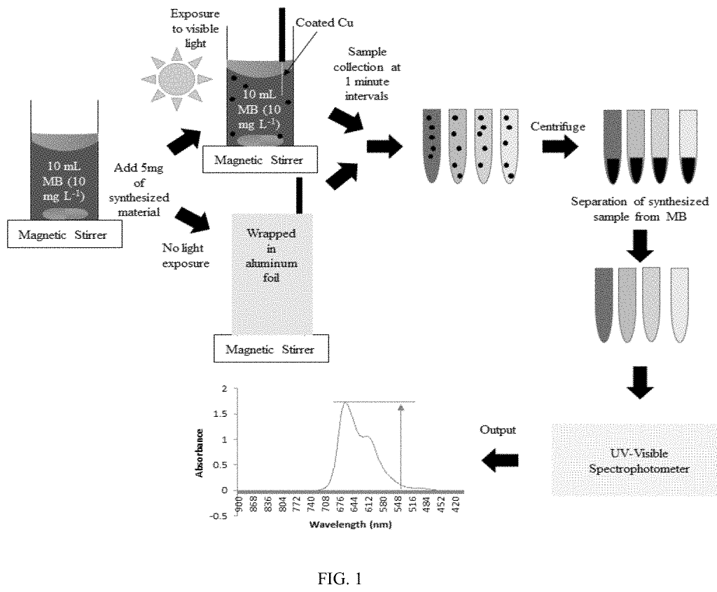

Water Decontamination Setup. Samples were suspended in 10 mL of a methylene blue solution that was continuously stirred, with a concentration of 10 mg L⁻¹. One set of experiments were conducted with exposure to visible light in the form of a 30 watt light with an intensity of 800 W/m², and another set were conducted without exposure to visible light, as shown in FIG. 1.

### Example 1. Synthesis of Molybdenum Dioxide (MoO₂) onto a Copper Substrate

To begin the experiment, a 1×1 cm (0.25 mm thick) 99.9% pure copper substrate was treated in hydrochloric acid for 15 min, followed by an ultrasonic bath in ethanol for 5 min. Next, 7.5 mL of deionized (DI) water and 2.5 mL of ethylene glycol were magnetically stirred, while 75 mg of MoO₃ powder was added. After 10 minutes of mixing, the solution was placed into a teflon-lined stainless steel pressure vessel along with the clean copper substrate. The pressure vessel was then sealed and placed into an oven at 180° C. 12 hours. After allowing the pressure vessel to naturally cool overnight and reach room temperature, the resulting solution was emptied into a beaker to retrieve the copper substrate. The copper substrate was then rinsed three times with DI water and ethanol before being placed in an oven to dry overnight. The weight of the copper before coating was approximately 250.5 mg, and after coating was approximately 252.5 mg.

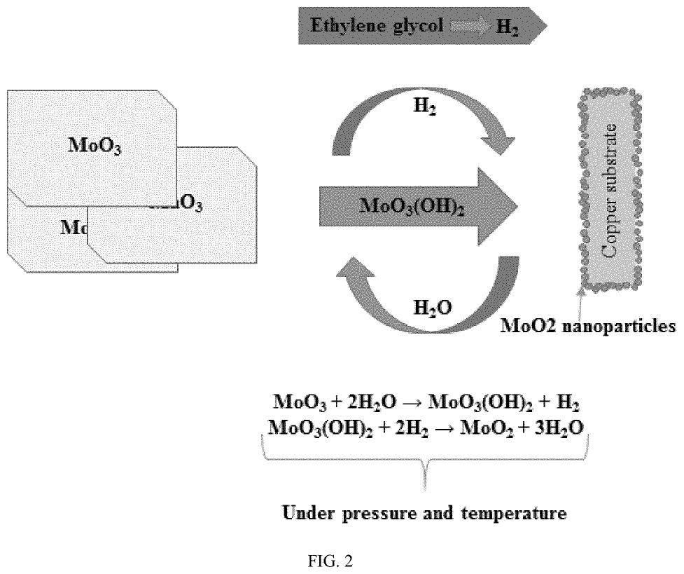

With being limited by any particular hypothesis, a schematic of the formation mechanism of MoO₂ nanoparticles directly onto a copper substrate is shown in FIG. 2. MoO₃, ethylene glycol, and water react under temperature and pressure to produce MoO₃(OH)₂, which is a volatile vapor phase that condensed onto the copper substrate, and subsequently dehydrated to form MoO₂.

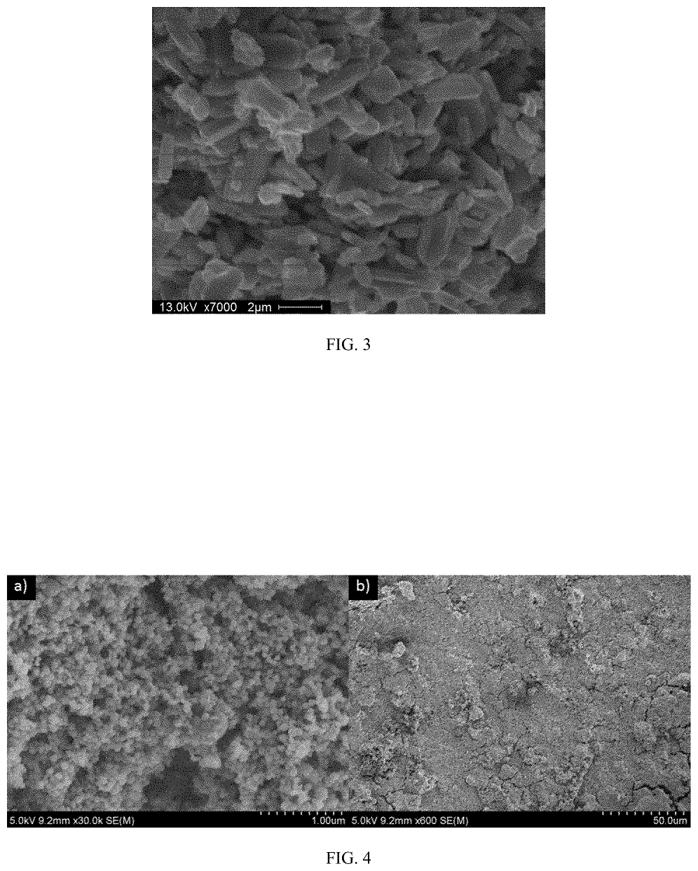

The samples were characterized by X-ray diffraction (XRD), scanning electron microscopy (SEM) and UV-visible spectrophotometry. FIG. 3 shows SEM images of the MoO₃ precursor powder with its platelet type structure. It is clear from FIG. 3 that the MoO₃ consists of large (>2 μm) platelet shaped particles. FIG. 4 shows the resulting MoO₂ nanoparticle coated on a copper substrate. It is clear that the synthesized MoO₂ coating consists of nanoparticles approximately 30-50 nm in diameter. There are clearly no larger MoO₃ pieces could be found anywhere on the samples, indicating all of the MoO₃ platelets had converted to MoO₂ nanoparticles, as later confirmed by the XRD analysis.

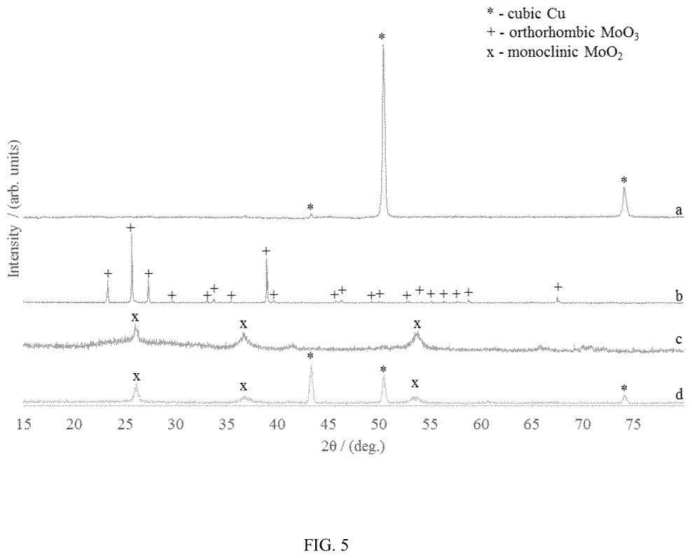

The MoO₂ coated copper substrate was analyzed using grazing incident angle X-ray diffraction (GIXRD). The coated samples were scanned with a fixed incident angle of 1°, while the pure Cu and MoO₃ were scanned using regular powder diffraction mode. FIG. 5 shows the XRD patterns for the materials used in this experiment. It should be noted that the patterns are not displayed at the same scale for clarity. It is clear from FIG. 5 that the synthesized films show a completely different XRD pattern when compared to the MoO₃ precursor. The coated samples show no indication of MoO₃ peaks, indicating a full conversion of MoO₃ to MoO₂. The MoO₂ coated sample had diffraction peaks at 26.1°, 36.8°, 43.4°, 50.5°, 53.3° and 74.1°. The diffraction peaks at 26.1°, 36.8°, and 53.3° correspond to the (−111), (200) and (022) planes of monoclinic MoO₂, respectively. The diffraction peaks seen at 43.4°, 50.5° and 74.1° are from the Cu substrate, and correspond to the (111), (200), and (220) planes of cubic copper, respectively.

### Example 2. Water Decontamination

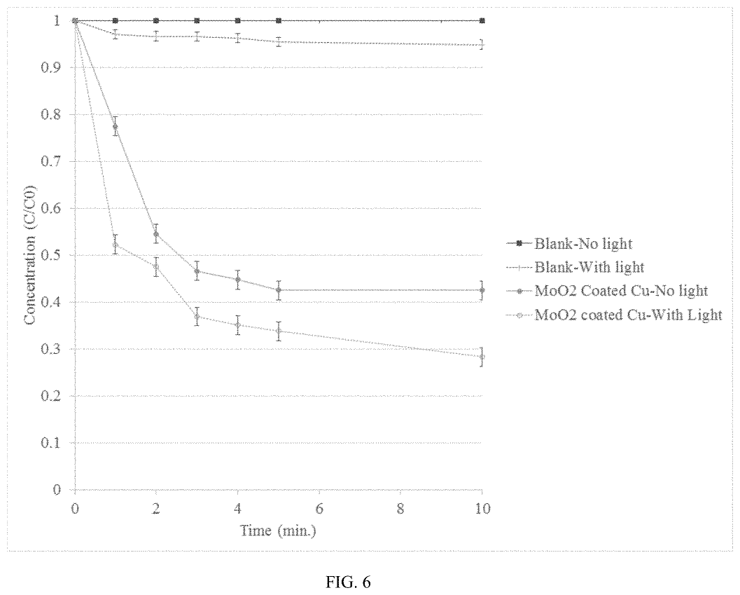

To determine the ability of the MoO₂ coated copper samples to decontaminate water, water decontamination experiments were carried out as described herein, and the results are shown in FIG. 6. It is clear from the image that the MoO₂ coated copper substrate is very effective at decontaminating MB from water. The MB degraded less than 0.05% during 10 minutes with no light exposure, and degraded 5.1% with exposure to light for 10 minutes. The coated samples were able to adsorb 57.5% of the MB with no exposure to light, while it was able to decontaminate 71.7% of the MB with light exposure.

As described herein, MoO₂ nanoparticles were successfully synthesized onto a copper substrate for the first time, as proven by XRD and SEM. The MoO₂ coated copper substrates were then tested for their ability to decontaminate MB from water. The MoO₂ coated copper substrates were not able to remove 100% of the MB, however it still was able to decontaminate over 50% of the MB from the water in 10 minutes with no light exposure, and over 70% removed in 10 minutes with light exposure.

For reasons of completeness, various aspects of the invention are set out in the following numbered clauses:

Clause 1. A method of synthesizing MoO₂ nanoparticles, comprising

mixing MoO₃, a metal substrate, and a reducing agent in water to form a mixture; and

heating the mixture, whereby MoO₂ nanoparticles are produced as a coating on the surface of the metal substrate.

Clause 2. The method of clause 1, wherein the metal substrate comprises copper, aluminum, nickel, titanium, steel, or a combination thereof.

Clause 3. The method of clause 1, wherein the metal substrate comprises copper.

Clause 4. The method of clause 1, wherein the reducing agent is ethylene glycol.

Clause 5. The method of clause 1, wherein the mixture is heated under a pressure greater than about 1 atm.

Clause 6. The method of clause 5, wherein the mixture is heated to about 180° C.

Clause 7. The method of clause 6, wherein the mixture is heated for less than or equal to about 12 hours.

Clause 8. The method of clause 1, wherein the MoO₂ nanoparticles have a diameter of about 30 nm to about 50 nm.

Clause 9. A product comprising a metal surface coated by MoO₂ nanoparticles according to the method of clause 1.

Clause 10. The product of clause 9, wherein the metal surface forms at least a part of an anode of a Li-ion battery, or wherein the metal surface forms at least a part of a symmetric or asymmetric electrode of a supercapacitor, or wherein the metal surface forms at least a part of a sensor for detecting a molecule selected from the group consisting of nitrogen oxide, nitrous oxide, alcohol, sulphur oxide, and a combination thereof.

Clause 11. A method of decontaminating water, comprising

contacting contaminated water comprising a water pollutant with the product of clause 9, thereby reducing the amount of the water pollutant.

Clause 12. The method of clause 11, wherein the water pollutant comprises an organic compound, a biological contaminant, or a combination thereof.

Clause 13. The method of clause 12, wherein the water pollutant comprises an organic compound selected from the group consisting of chloroform, alcohols, trichloroacetic acid, 4-chlorophenol, pentachlorophenol, benzene, chlorobenzene, 4-chlorotoluene, or a combination thereof.

Clause 14. The method of clause 12, wherein the water pollutant comprises an organic compound selected from the group consisting of a chemical toxin, a dye, or a combination thereof.

Clause 15. The method of clause 14, wherein the dye is methylene blue.

Clause 16. The method of clause 12, wherein the water pollutant comprises a biological contaminant selected from the group consisting of a protein, a bacterium, or a combination thereof.

Clause 17. The method clause 16, wherein the bacterium is selected from the group consisting of E. coli, Campylobacter jejuni, Clostridium perfringens, Salmonella spp., Bacillus cereus, Listeria monocytogenes, Shigella spp., Staphylococcus aureus, Staphylococcal enteritis, Streptococcus, Vibrio cholera, Vibrio parahaemolyticus, Vibrio vulnificus, Yersinia enterocolitica, Yersinia pseudotuberculosis, and a combination thereof.

Clause 18. The method of clause 12, further comprising

carrying out a photocatalytic reaction of the water pollutant catalyzed by the MoO₂ coated surface under UV to visible light; and/or

absorbing the water pollutant onto the MoO₂ coated surface and oxidizing the water pollutant into carbon dioxide, water or a small molecule.

Clause 19. A method of decontaminating air, comprising

contacting contaminated air comprising an air pollutant with the product of clause 9, thereby reducing the amount of the air pollutant.

Clause 20. The method of clause 19, wherein the air pollutant comprises an organic gas, an inorganic gas, a biotoxin, or a combination thereof.

Various features and advantages of the invention are set forth in the following claims.

What is claimed is:

1. A method of synthesizing MoO₂ nanoparticles, comprising
mixing MoO₃, a metal substrate, and a reducing agent in water to form a mixture; and
heating the mixture, whereby MoO₂ nanoparticles are produced as a coating on the surface of the metal substrate.

2. The method of claim 1, wherein the metal substrate comprises copper, aluminum, nickel, titanium, steel, or a combination thereof.

3. The method of claim 1, wherein the metal substrate comprises copper.

4. The method of claim 1, wherein the reducing agent is ethylene glycol.

5. The method of claim 1, wherein the mixture is heated under a pressure greater than about 1 atm.

6. The method of claim 5, wherein the mixture is heated to about 180° C.

7. The method of claim 6, wherein the mixture is heated for less than or equal to about 12 hours.

8. The method of claim 1, wherein the MoO₂ nanoparticles have a diameter of about 30 nm to about 50 nm.

9. A product comprising a metal surface coated by MoO₂ nanoparticles according to the method of claim 1.

10. The product of claim 9, wherein the metal surface forms at least a part of an anode of a Li-ion battery, or wherein the metal surface forms at least a part of a symmetric or asymmetric electrode of a supercapacitor, or wherein the metal surface forms at least a part of a sensor for detecting a molecule selected from the group consisting of nitrogen oxide, nitrous oxide, alcohol, sulphur oxide, and a combination thereof.

11. A method of decontaminating water, comprising
contacting contaminated water comprising a water pollutant with the product of claim 9, thereby reducing the amount of the water pollutant.

12. The method of claim 11, wherein the water pollutant comprises an organic compound, a biological contaminant, or a combination thereof.

13. The method of claim 12, wherein the water pollutant comprises an organic compound selected from the group consisting of chloroform, alcohols, trichloroacetic acid, 4-chlorophenol, pentachlorophenol, benzene, chlorobenzene, 4-chlorotoluene, or a combination thereof.

14. The method of claim 12, wherein the water pollutant comprises an organic compound selected from the group consisting of a chemical toxin, a dye, or a combination thereof.

15. The method of claim 14, wherein the dye is methylene blue.

16. The method of claim 12, wherein the water pollutant comprises a biological contaminant selected from the group consisting of a protein, a bacterium, or a combination thereof.

17. The method claim 16, wherein the bacterium is selected from the group consisting of E. coli, Campylobacter jejuni, Clostridium perfringens, Salmonella spp., Bacillus cereus, Listeria monocytogenes, Shigella spp., Staphylococcus aureus, Staphylococcal enteritis, Streptococcus, Vibrio cholera, Vibrio parahaemolyticus, Vibrio vulnificus, Yersinia enterocolitica, Yersinia pseudotuberculosis, and a combination thereof.

18. The method of claim 12, further comprising
carrying out a photocatalytic reaction of the water pollutant catalyzed by the MoO₂ coated surface under UV to visible light; and/or
absorbing the water pollutant onto the MoO₂ coated surface and oxidizing the water pollutant into carbon dioxide, water or a small molecule.

19. A method of decontaminating air, comprising
contacting contaminated air comprising an air pollutant with the product of claim 9, thereby reducing the amount of the air pollutant.

20. The method of claim 19, wherein the air pollutant comprises an organic gas, an inorganic gas, a biotoxin, or a combination thereof.
